(19) United States
(12) Patent Application Publication
(10) Pub. No.: US 2026/0051047 A1
(43) Pub. Date: Feb. 19, 2026

(54) LESION MAPPING ASSISTANT SYSTEM AND METHOD

(71) Applicant: Thalocan Research Innovations LLC, Indianapolis, IN (US)

(72) Inventors: Eric ZUDAK, Indianapolis, IN (US); Cyrus MIRAKHOR, Indianapolis, IN (US); Paul STROILI, Skokie, IL (US)

(21) Appl. No.: 18/805,248

(22) Filed: Aug. 14, 2024

(51) Int. Cl. G06T 7/00; G16H 30/40

(52) U.S. Cl. CPC G06T 7/0012; G16H 30/40; G06T 2207/30096

(57) ABSTRACT

A method for mapping one or more cutaneous conditions of a cutaneous surface may include: receiving, by at least one processor, a captured representation of a cutaneous surface having one or more cutaneous conditions and one or more measurement markers; defining, by the at least one processor, one or more boundaries around the one or more cutaneous conditions on the captured representation to form one or more enclosed areas; determining, by the at least one processor and based on a comparison between the one or more measurement markers and the one or more enclosed areas, characteristics of the one or more cutaneous conditions enclosed within the one or more enclosed areas; and rendering, by the at least one processor on at least one display, the captured representation and the determined characteristics of the one or more cutaneous conditions enclosed within the one or more enclosed areas.

### BACKGROUND

### 1. Field

Aspects of the present disclosure relate to a system and method for mapping one or more cutaneous conditions of a cutaneous surface.

### 2. Description of the Related Art

In the field of dermatology, present methods of mapping cutaneous conditions, such as lesions, nodules, papules, discolored patches, and other relevant cutaneous abnormalities, face several limitations. Typically, such methods rely on visual assessments of the affected area, which are subjective, and prone to human error. Further, notes of such visual assessments are often recorded manually, for e.g., via pen and paper, which can lack in detail and nuance. Moreover, the characteristics (e.g., size, shape, count, location, color, etc.) of cutaneous conditions continuously change over time. Consequently, present methods may overlook subtle changes in these characteristics, which may result in inconsistent and inaccurate diagnosis and treatment.

Moreover, many diseases use the percentage of involvement as a severity criteria. The clinician will estimate the percentage by observing a bodily region (trunk, lower extremities, head, etc., to determine the extent of involvement. The various current methods used for such estimation are not precise and introduce a high degree of subjectivity to the calculation. Further if the assessment is made by more than one person during the course of a study, the question of differences in the subjective estimation introduces further uncertainty into the reliability of any assessment of the ongoing progression of the disease.

Further, placebo controls in research studies directed to the study of cutaneous conditions, are negatively impacted by the shortcomings of such methods of visual assessments. For example, because such methods of visual assessment are conducted in a subjective and disjointed manner, they may over count or under count the occurrence of cutaneous conditions, leading to inaccurate measurement of placebo effects. Also, placebo control groups may be analyzed inconsistently during the course of research studies. This may further lead to inaccurate placebo control outcomes, thereby jeopardizing the entirety of the research studies.

Additionally, the lack of an accessible central repository of past assessments of the affected area may result in further inconsistency and inaccuracy, especially if different healthcare providers are involved in diagnosis and treatment. Accordingly, a need exists for improving present methods of assessing cutaneous conditions. It is with respect to this need, that aspects of the present disclosure are directed.

The above information disclosed in this Background section is for enhancement of understanding of the background of the present disclosure, and, therefore, it may contain information that does not constitute prior art.

### SUMMARY

According to an aspect, a method for mapping one or more cutaneous conditions of a cutaneous surface may include: receiving, by at least one processor, a captured representation of a cutaneous surface having one or more cutaneous conditions and one or more measurement markers; defining, by the at least one processor, one or more boundaries around the one or more cutaneous conditions on the captured representation to form one or more enclosed areas; determining, by the at least one processor and based on a comparison between the one or more measurement markers and the one or more enclosed areas, characteristics of the one or more cutaneous conditions enclosed within the one or more enclosed areas; and rendering, by the at least one processor on at least one display, the captured representation and the determined characteristics of the one or more cutaneous conditions enclosed within the one or more enclosed areas.

According to another aspect, a system for mapping one or more cutaneous conditions of a cutaneous surface may comprise: at least one processor; at least one display, the at least one display configured to receive user input, and operatively connected to the at least one processor; at least one camera, the at least one camera operatively connected to the at least one processor; and a memory, the memory operatively connected to the at least one processor and storing instructions that, when executed, cause the at least one processor to perform a method, the method comprising: receiving a captured representation of a cutaneous surface having one or more cutaneous conditions and one or more measurement markers; defining one or more boundaries around the one or more cutaneous conditions on the captured representation to form one or more enclosed areas; determining, based on a comparison between the one or more measurement markers and the one or more enclosed areas, characteristics of the one or more cutaneous conditions enclosed within the one or more enclosed areas; and rendering, on the at least one display, the captured representation and the determined characteristics of the one or more cutaneous conditions enclosed within the one or more enclosed areas.

According to another aspect, a system for mapping one or more cutaneous conditions of a cutaneous surface may comprise: at least one processor; at least one display, the at least one display configured to receive user input, and operatively connected to the at least one processor; the at least one remote sensor operatively connected to the at least one processor; and a memory, the memory operatively connected to the at least one processor and storing instructions that, when executed, cause the at least one processor to perform a method, the method comprising: receiving a captured representation of a cutaneous surface having one or more cutaneous conditions and one or more measurement markers; defining one or more boundaries around the one or more cutaneous conditions on the captured representation to form one or more enclosed areas; determining, based on a comparison between the one or more measurement markers and the one or more enclosed areas, characteristics of the one or more cutaneous conditions enclosed within the one or more enclosed areas; and rendering, on the at least one display, the captured representation and the determined characteristics of the one or more cutaneous conditions enclosed within the one or more enclosed areas.

In an example, the one or more measurement markers may comprise physical markers having predetermined characteristics (e.g., size, shape, color, etc.). The predetermined characteristics, for e.g., the size, of a first measurement marker of the one or more measurement markers may be different from the size of a second measurement marker of the one or more measurement markers. The one or more measurement markers may be manually placed by a user, for e.g., a clinician, on a cutaneous surface having one or more cutaneous conditions to be mapped. The manual placement may be accomplished through the use of one or more physical stickers having an adhesive backing able to adhere to the cutaneous surface.

In another example, the manual placement may be accomplished through use of a permanent marking, such as a tattoo, and/or semi-permanent marking, such as with an indelible marker, made on the cutaneous surface.

In another example, the manual placement may be accomplished by including in the representation one or more naturally occurring distinctive markings that are likely to remain unchanged over time, such as moles, freckles or body parts, such as a nipple or navel. Combinations of the ways of manual placement described herein may also be used where more than one measurement marker is used.

In another example, the captured representation may comprise an image that may be captured by at least one camera operatively connected to the at least one processor.

In another example, the captured representation may comprise a topographic image that may be captured by at least one remote sensor operatively connected to the at least one processor.

In another example, the defining may include defining, by at least one processor, one or more boundaries, which may be virtual or physical, on the captured representation based on user input from at least one display operatively connected to the at least one processor.

In another example, the defining may further include the manual placing by a user, for e.g., a clinician, of one or more boundaries respectively enclosing the one or more cutaneous conditions on the captured representation to form one or more enclosed areas on the display.

In another example, the set of instructions stored on a memory operatively connected to the at least one processor may cause the processor to align the one or more boundaries to correspond to the edges of the one or more cutaneous conditions respectively enclosed within the one or more enclosed areas.

In another example, the determining may include identifying a first set of characteristics corresponding to a measurement marker of the one or more measurement markers and identifying a second set of characteristics corresponding to an enclosed area of the one or more enclosed areas.

In another example, the determining may further include comparing the first set of characteristics respectively with the second set of characteristics.

In another example, the determining may include identifying a third set of characteristics based on the comparison between the first set of characteristics and the second set of characteristics, the third set of characteristics corresponding to the cutaneous condition within the enclosed area.

In another example, the rendering may include rendering the captured representation on the at least one display.

In another example, the rendering may include rendering a visual representation of the third set of characteristics corresponding to the cutaneous condition within the enclosed area on the at least one display.

In another example, the rendering may further include rendering a portion of the captured representation on the at least one display, the portion corresponding to the cutaneous condition within the enclosed area.

In another example, the rendering may further include rendering one or more portions of previously captured representations of the cutaneous condition within the enclosed area on the at least one display.

In another example, the rendering may include rendering one or more visual representations of previously determined sets of characteristics corresponding to the cutaneous condition within the enclosed area on the at least one display.

In another example, a set of characteristics may be communicated, by the at least one processor, to a cloud database for storage.

In another example, a set of characteristics stored in the cloud database may be accessed by the at least one memory operatively connected to the at least one processor.

In another example, a set of characteristics stored in the cloud database may be accessed by a first processor located in a first location and a second processor located in the first location.

In another example, a set of characteristics stored on the cloud database may be accessed by a first processor located in a first location and a second processor located in a second location.

This summary is provided to introduce a selection of features and concepts of example embodiments of the present disclosure that are further described below in the detailed description. This summary is not intended to identify key or essential features of the claimed subject matter nor is it intended to be used in limiting the scope of the claimed subject matter. One or more of the described features according to one or more examples may be combined with one or more other described features according to one or more examples to provide a workable method or device.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings, together with the specification, illustrate non-limiting and non-exhaustive examples of the present disclosure.

FIG. 1 depicts a frontal view of an example cutaneous surface having one or more measurement markers.

FIG. 2 depicts a block diagram including an example system for mapping one or more cutaneous conditions of a cutaneous surface.

FIG. 3 depicts a flowchart including an example method to receive, by at least one processor, a captured representation of a cutaneous surface having one or more cutaneous conditions and one or more measurement markers.

FIG. 4 depicts a flowchart including an example method to define, by the at least one processor, one or more boundaries around the one or more cutaneous conditions on the captured representation to form one or more enclosed areas.

FIG. 5 depicts a flowchart including an example method to determine, by the at least one processor and based on a comparison between the one or more measurement markers and the one or more enclosed areas, characteristics of the one or more cutaneous conditions enclosed within the one or more enclosed areas.

FIG. 6 depicts a flowchart including an example method for rendering, by the at least one processor on at least one display, the captured representation and the determined characteristics of the one or more cutaneous conditions enclosed within the one or more enclosed areas.

FIG. 7 depicts the captured representation of FIG. 3 according to some examples.

FIG. 8 depicts the one or more boundaries forming the one or more enclosed areas of FIG. 4 according to some examples.

FIGS. 9 and 10 depict the captured representation and the determined characteristics as described in FIG. 6 according to some examples.

FIG. 11 depicts interactive prompts generated by the LMA according to some examples.

FIG. 12 depicts a ratio generated by the LMA according to some examples.

### DETAILED DESCRIPTION

Features of the inventive concept and methods of accomplishing the same may be understood more readily by reference to the following detailed description of example embodiments and the accompanying drawings. Hereinafter, example embodiments will be described in more detail with reference to the accompanying drawings. The present invention, however, may be embodied in various different forms, and should not be construed as being limited to only the illustrated example embodiments herein. Rather, these example embodiments are provided as examples so that this disclosure will be thorough and complete, and will fully convey the aspects and features of the present invention to those skilled in the art. Accordingly, processes, elements, and techniques that are not necessary to those having ordinary skill in the art for a complete understanding of the aspects and features of the present invention may not be described. Unless otherwise noted, like reference numerals denote like elements throughout the attached drawings and the written description, and thus, descriptions thereof will not be repeated. Further, parts not related to the description of the example embodiments might not be shown to make the description clear. In the drawings, the relative sizes of elements, layers, and regions may be exaggerated for clarity.

In the following description, for the purposes of explanation, numerous specific details are set forth to provide a thorough understanding of various example embodiments. It is apparent, however, that various example embodiments may be practiced without these specific details or with one or more equivalent arrangements.

The terminology used herein is for the purpose of describing particular example embodiments only and is not intended to be limiting of the present invention. As used herein, the singular forms “a” and “an” are intended to include the plural forms as well, unless the context clearly indicates otherwise. It will be further understood that the terms “comprises,” “comprising,” “have,” “having,” “includes,” and “including,” when used in this specification, specify the presence of the stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, the term “substantially,” “about,” “approximately,” and similar terms are used as terms of approximation and not as terms of degree, and are intended to account for the inherent deviations in measured or calculated values that would be recognized by those of ordinary skill in the art. “About” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value. Further, the use of “may” when describing example embodiments of the present disclosure refers to “one or more example embodiments of the present disclosure.” As used herein, the terms “use,” “using,” and “used” may be considered synonymous with the terms “utilize,” “utilizing,” and “utilized,” respectively. Also, the term “exemplary” is intended to refer to an example or illustration.

When a certain example embodiment may be implemented differently, a specific process order may be performed differently from the described order. For example, two consecutively described processes may be performed substantially at the same time or performed in an order opposite to the described order.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

Systems and methods for mapping one or more cutaneous conditions of a cutaneous surface will now be described herein with reference to the figures.

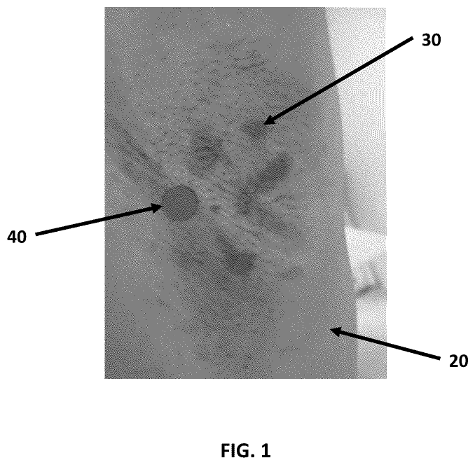

FIG. 1 depicts a frontal view of an example cutaneous surface 20 having one or more measurement markers according to one or more examples of the present disclosure. In an illustrative example, the cutaneous surface 20 may include multiple measurement markers, including the measurement marker 40.

Referring to FIG. 1, the cutaneous surface may further include one or more cutaneous conditions. In an illustrative example, the cutaneous surface 20 may include multiple cutaneous conditions, including the cutaneous condition 30.

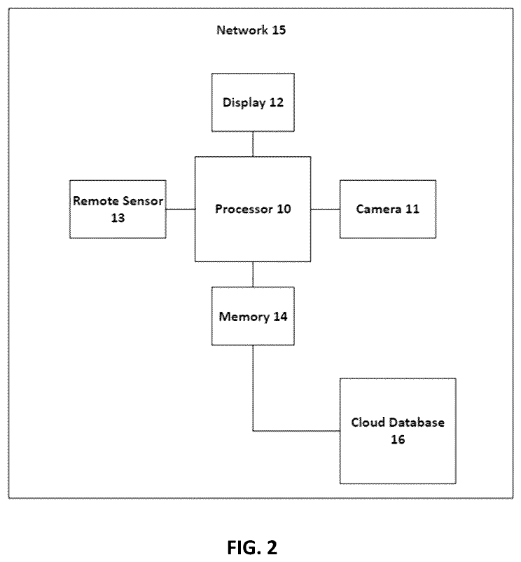

FIG. 2 depicts a block diagram illustrating an example system for mapping one or more cutaneous conditions of a cutaneous surface of the cutaneous surface.

Referring to FIG. 2, the system may include a processor 10 that may be operatively connected to a memory 14. For example, the memory 14 may be operatively connected to the processor 10 via a network 15. In other examples, the processor 10 and the memory 14 may be integrated as a single system (for e.g., as a personal cellular device).

As shown in FIG. 2, the processor 10 may be operatively connected to a camera 11. For example, the camera 11 may be operatively connected to the processor 10 via a network 15. In further examples, the processor 10, the memory 14 and the camera 11, or any combination thereof, may be integrated as a single system (for e.g., as a personal cellular device).

In another example, the camera 11 may be configured to capture a representation, for example, a visual representation (for e.g., a digital image) of the cutaneous surface 20 when executing instructions stored in the memory 14.

In another example, the camera 11 may include at least one processor and at least one memory that may be operatively connected to the processor 10 and the memory 14. For example, the at least one processor and at least one memory of the camera 11 may be operatively connected to the processor 10 and the memory 14 via the network 15.

In some examples, the at least one processor of the camera 11 may be configured to store a visual representation captured by the camera 11 in the at least one memory of the camera 11. For example, the visual representation captured by the camera 11 may be stored (for e.g., temporarily stored) in the at least one memory of the camera 11. In further examples, the at least one processor of the camera 11 may communicate the visual representation captured by the camera 11 stored in the at least one memory of the camera 11 to the memory 14, for example, via the network 15.

As shown in FIG. 2, the processor 10 may be operatively connected to a display 12. For example, the display 12 may be configured to communicate with the processor 10 via the network 15. In other examples, the processor 10, the memory 14, the camera 11 and the display 12, or any combination thereof, may be integrated as a single system (for e.g., as a personal cellular device). The processor 10 may include the display 12 and may also include other output device(s) such as speakers, a printer, etc. The aforementioned devices are examples and others may be used.

In another example, the display 12 may be configured to receive user input (for e.g., a capacitive touch display). In other examples, the processor 10 may further be operatively connected to other input devices such as a keyboard, a mouse, a pen, a sound input device, a touch input device, etc.

In another example, the processor 10 may store the user input received by the display 12 in the memory 14.

As shown in FIG. 2, the processor 10 may be operatively connected to a remote sensor 13. For example, the remote sensor 13 may be operatively connected to the processor 10 via the network 15. In further examples, the processor 10, the memory 14, the camera 11, the display 12 and the remote sensor 13, or any combination thereof, may be integrated as a single system (for e.g., as a personal cellular device).

In another example, the remote sensor 13 may be configured to detect characteristics (for e.g., height and density) of the cutaneous condition 30 and/or the measurement marker 40 present on the cutaneous surface 20. For example, the remote sensor 13 may be a light detection and ranging (LiDAR) sensor, which may be used to compile a 3-D digital map of the cutaneous surface 20.

In another example, the remote sensor 13 may be configured to capture a representation, for example, a topographic representation (for e.g., a three-dimensional digital image) of the cutaneous surface 20 when executing instructions stored in the memory 14.

In some examples, the remote sensor 13 may include at least one processor and at least one memory that may be operatively connected to the processor 10 and the memory 14. For example, the at least one processor of the remote sensor 13 may be configured to store a topographic representation captured by the remote sensor 13 in the at least one memory of the camera 11. In further examples, the topographic representation captured by the remote sensor 13 may be stored (for e.g., temporarily stored) in the at least one memory of the remote sensor 13. In other examples, the at least one processor of the remote sensor 13 may communicate the topographic representation captured by the remote sensor 13 stored in the at least one memory of the remote sensor 13 to the memory 14, for example, via the network 15.

As shown in FIG. 2, the memory 14 may be configured to communicate with the processor 10 via the network 15. In some examples, the memory 14 may comprise a volatile storage (for e.g., random access memory), non-volatile storage (for e.g., read-only memory), flash memory, or any combination thereof. The memory 14 may be configured to store a one or more representations captured by the camera 11. The memory 14 may further be configured to store one or more topographic representations captured by the remote sensor 13.

In another example, the processor 10 and/or the memory 14 may be communicatively coupled to other processors and/or other memory devices via a communication medium, such as via the network 15. In some examples, the communication medium may include, without limitation, RF transmitter, receiver, and/or transceiver circuitry; universal serial bus (USB), parallel, and/or serial ports, or any combination thereof.

In another example, the memory 14 and/or the processor 10 may further be communicatively coupled with a cloud database 16. For example, the memory 14 may be communicatively coupled with a cloud database 16 via the network 15. In other examples, the memory 14 may be communicatively coupled with a cloud database 16 via any applicable wireless and/or wired communication means.

In some examples, the processor 10 may communicate data stored in the memory 14 to the cloud database 16. The cloud database 16 may store the communicated data.

In another example, the processor 10 may receive data from the cloud database 16 to store in the memory 14.

In another example, the memory 14 may further be configured to store one or more software applications such as a Lesion Mapping Assistant (the “LMA”) 500. The LMA 500 may include instructions that, when executed by the processor 10, cause the system to perform the methods described herein to determine characteristics of one or more cutaneous conditions, including the cutaneous condition 30 present on the cutaneous surface 20.

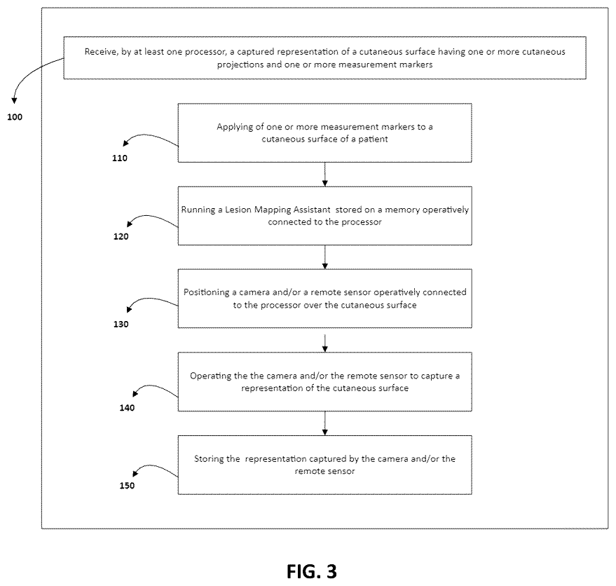

FIG. 3 depicts a flowchart including an example method 100 to receive, by at least one processor, a captured representation of a cutaneous surface having one or more cutaneous conditions and one or more measurement markers. The method 100 may include a first process 110 of applying one or more measurement markers on a cutaneous surface of a patient. In an illustrative example, the measurement marker 40 may be placed by a user, for e.g., a clinician, onto the cutaneous surface 20 having the cutaneous condition 30. This may be accomplished by including an adhesive backing on a sticker that allows it to be adhered to the skin at least temporarily. Alternatively, the placement of a marking on the cutaneous surface 20 may be accomplished through use of a permanent marking technique, such as a tattoo, or through use of a semi-permanent marking technique, such as through use of an indelible marker. Advantage may also be taken of naturally occurring distinctive features on the cutaneous surface 20 that are likely to remain unchanged, such as moles, freckles, or body parts like a nipple or navel, if those distinctive features are located near the cutaneous surface under examination. If more than one measurement marker is to be used, a combination of these various types of markers may be employed.

Referring to FIG. 3, the method 100 may include a second process 120 of running the LMA stored on a memory operatively connected to a processor. In an illustrative example, the LMA 500 may be stored on the memory 14 operatively connected to the processor 10. In further examples, the processor 10 may be caused to run the LMA 500 by a user, for e.g., a clinician.

In another example, the method 100 may include a third process 130 of positioning a camera and/or a remote sensor over the cutaneous surface. In an illustrative example, the camera 11 and/or the remote sensor 13 may be positioned over the cutaneous surface by a user, for e.g., a clinician, so that the measurement marker 40 and the cutaneous condition 30 are within the capturing area of the camera 11 and/or the remote sensor 13.

In another example, the method 100 may include a fourth process 140 of operating the camera and/or the remote sensor to capture a representation of the cutaneous surface having the one or more measurement markers. In an illustrative example, the camera 11 and/or a remote sensor 13 may be operated by a user, for e.g., a clinician, to capture a representation of the cutaneous surface 20 having the measurement marker 40 and the cutaneous condition 30.

In another example, the method 100 may include a fifth process 150 of storing the representation captured by the camera and/or the remote sensor. In an illustrative example, the captured representation 50 captured by the camera 11 and/or the remote sensor 13 may be stored, for e.g., in the memory 14 via the processor 10.

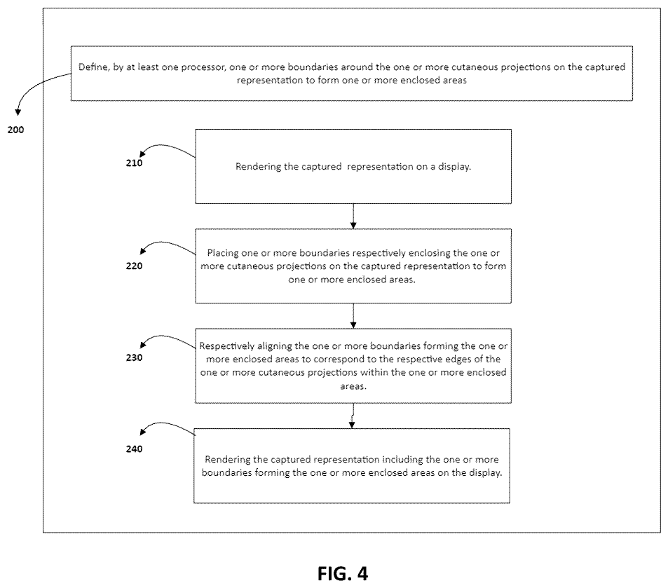

FIG. 4 depicts a flowchart including an example method 200 to define, by at least one processor, one or more boundaries around the one or more cutaneous conditions on the captured representation to form one or more enclosed areas. The method 200 may include a first process 210 of rendering the captured representation on a display. In an illustrative example, the captured representation 50 may be rendered, for example, on the display 12.

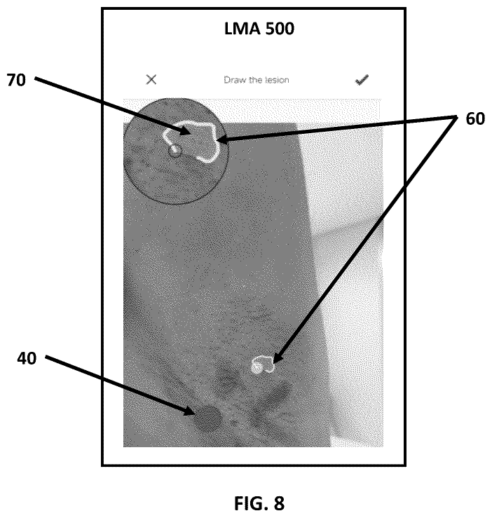

Referring to FIGS. 4 and 8, the method 200 may include a second process 220 of placing one or more boundaries respectively enclosing the one or more cutaneous conditions on the captured representation to form one or more enclosed areas. In further examples, the one or more cutaneous conditions may be respectively enclosed within the one or more enclosed areas.

In an illustrative example, the LMA 500 may allow a user, for e.g., a clinician, to interact with the display 12 (for e.g., via a capacitive-touch based display) to place a boundary 60 around the cutaneous condition 30. The user may input the boundary 60 on the display 12, for e.g., via placing their finger(s) on the display, such that the boundary corresponds to the edges of the cutaneous condition 30 rendered on the display 12.

In another example, the placement of the boundary 60 may be accomplished physically through use of a marking technique, such as through use of a semi-permanent marking technique, such as through use of an indelible marker on the cutaneous surface 20.

In another example, the method 200 may include a third process 230 of aligning the one or more boundaries forming the one or more enclosed areas to correspond to the edges of the one or more cutaneous conditions within the one or more enclosed areas. In an illustrative example, the LMA 500 may cause the processor 10 to align the boundary 60 to correspond to the edges of the cutaneous condition 30 within the enclosed area 70.

In another example, the second process 220 and the third process 230 may be performed by the LMA contemporaneously and/or simultaneously. For example, the user may interact with the at least one display to place one or more points on the captured representation that correspond to the edges of a cutaneous condition of the one or more cutaneous conditions. The LMA may then, based on the one or more points placed by the user, form a boundary on the captured representation to enclose the cutaneous condition.

In another example, the method 200 may include a fourth process 240 of rendering the captured representation including the one or more boundaries forming the one or more enclosed areas on the display. In an illustrative example, the captured representation 50 including the boundary 60 may be rendered on the display 12. In further examples, the captured representation 50 including the boundary 60 may be contemporaneously rendered on the display 12 while the user manually places the boundary 60 around the cutaneous condition 30.

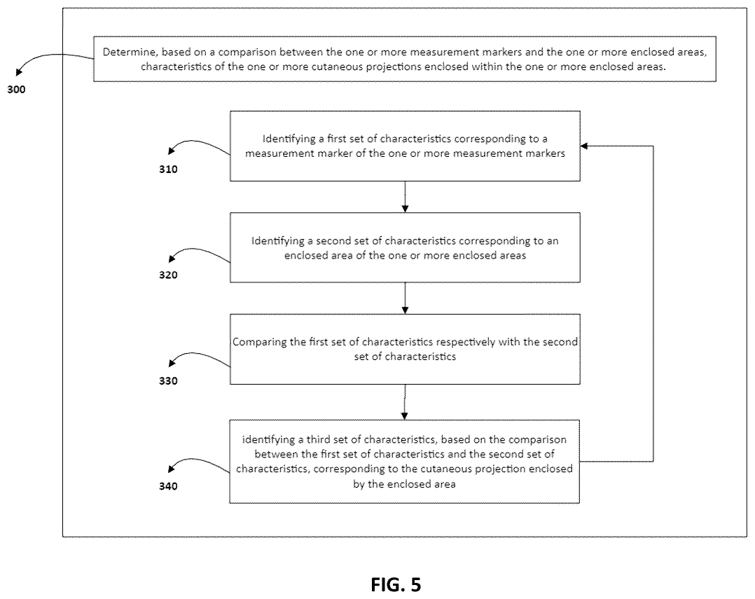

FIG. 5 depicts a flowchart including an example method 300 to determine, based on a comparison between the one or more measurement markers and the one or more enclosed areas, characteristics of the one or more cutaneous conditions enclosed within the one or more enclosed areas. That is, characteristics of the one or more cutaneous conditions may be determined by comparing those characteristics for the cutaneous conditions to those same characteristics for the measurement markers, those characteristics for the measurement markers being a known value or quantity.

Referring to FIG. 5, the method 300 may include a first process 310 of identifying a first set of characteristics corresponding to a measurement marker of the one or more measurement markers. The first set of characteristics may comprise quantitative and/or qualitative values of the size, shape, color, or any other applicable characteristics of the measurement marker. In an illustrative example, the LMA 500 may cause the processor 10 to determine a first set of characteristics of the measurement marker 40. This may allow the LMA to establish a correlation between the known characteristics of the measurement markers and how those characteristics are measured by the system to establish one or more reference points for the system.

In further examples, the LMA, via the at least one processor and the at least one display, may allow a user of the LMA to change quantitative and/or qualitative values of the size, shape, color, or any other applicable characteristics of a measurement marker of the one or more measurement markers. In an illustrative example, a user of the LMA 500, for e.g., a clinician, may interact with the display 12 to manually change the quantitative and/or qualitative values of the first set of characteristics that correspond to the measurement marker 40 determined by the processor 10.

In another example, the method 300 may include a second process 320 of identifying a second set of characteristics corresponding to an enclosed area of the one or more enclosed areas. The second set of characteristics may comprise quantitative and/or qualitative values of the size, shape, color, or any other applicable characteristics of the enclosed area. In an illustrative example, the LMA may cause the processor 10 to identify a second set of characteristics corresponding to the enclosed area 70.

In another example, the method 300 may include a third process 330 of comparing the first set of characteristics with the second set of characteristics.

In another example, the method 300 may include a fourth process 340 of identifying a third set of characteristics based on the comparison between the first set of characteristics and the second set of characteristics, the third set of characteristics corresponding to the cutaneous condition enclosed by the enclosed area. The third set of characteristics may comprise quantitative and/or qualitative values of the size, shape, color, or any other applicable characteristics of the cutaneous conditions within the enclosed area. In an illustrative example, the LMA 500 may cause the processor 10 to identify a third set of characteristics corresponding to the cutaneous condition 30 within the enclosed area 70.

In another example, steps of the method 300 may be repeated separately and/or contemporaneously for each of the one or more cutaneous conditions enclosed by the one or more enclosed areas to respectively identify the corresponding one or more third sets of characteristics.

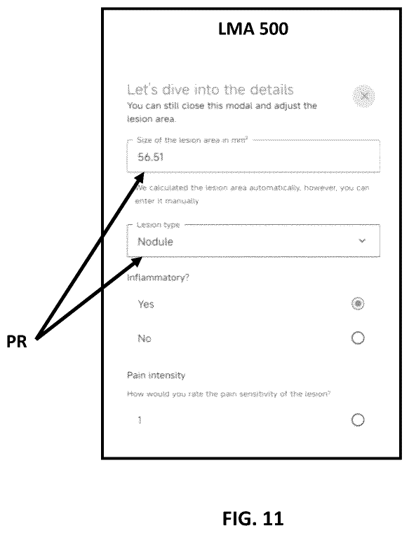

In another example, the LMA may allow a user to interact with the display, such that the user is able to change any information, including any values and/or quantities, of the determined sets of characteristics of the one or more lesions of the cutaneous surface. In an illustrative example, the LMA 500 may allow a user to interact with the display 12 to modify values and/or quantities of the determined third set of characteristics of the cutaneous condition 30, as depicted in FIG. 11.

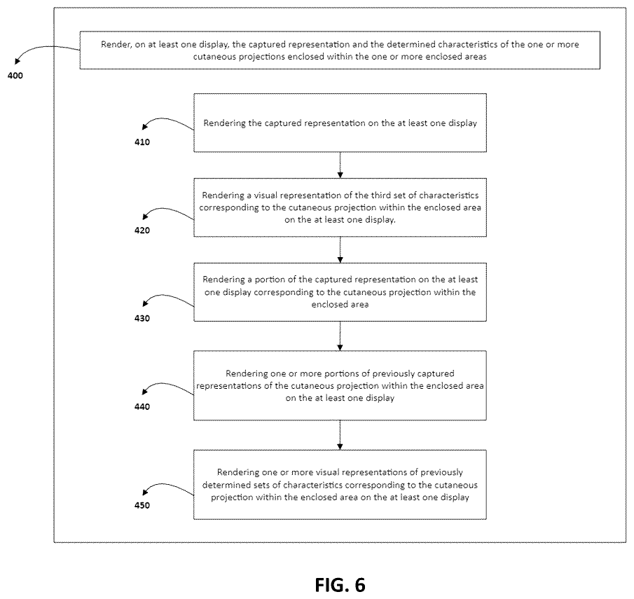

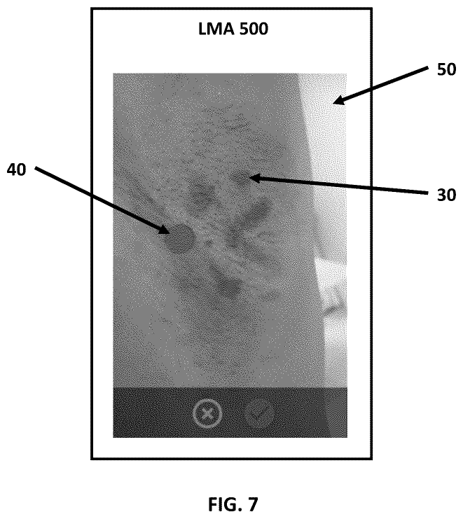

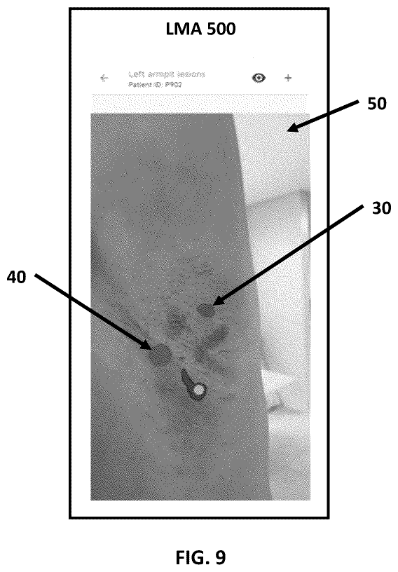

FIG. 6 depicts a flowchart including an example method 400 for rendering, on at least one display, the captured representation and the determined characteristics of the one or more cutaneous conditions enclosed within the one or more enclosed areas. Referring to FIGS. 6, 9, and 10, the method 400 may include a first process 410 of rendering the captured representation on the at least one display. In an illustrative example, the LMA 500 may render the captured representation 50 on the at least one display 12. The captured representation 50 rendered on the display 12 may include the boundary 60 and the enclosed area 70. In further examples, a user of the LMA may interact with the display 12 to view any sets of determined characteristics of the cutaneous condition 30 enclosed by the boundary 60.

In another example, the method 400 may include a second process 420 of rendering a visual representation of the third set of characteristics corresponding to the cutaneous condition within the enclosed area on the at least one display. In an illustrative example, the LMA 500 may render the third set of characteristics corresponding to the cutaneous condition 30. The LMA 500 may render the third set of characteristics based on user input received, for e.g., by the display 12. In further examples, the LMA 500 may allow the user to interact with the display 12 to modify any values and/or quantities of the third set of determined characteristics.

In another example, the method 400 may include a third process 430 of rendering a portion of the captured representation on the at least one display, the portion corresponding to the cutaneous condition within the enclosed area. For example, the LMA may render, on the at least one display, a portion of the captured representation of the cutaneous surface, the portion of the captured representation corresponding to the one or more cutaneous surfaces present on the captured representation. The LMA 500 may perform this operation based on user input received, for e.g., by the display 12. In an illustrative example, the LMA 500 may render a portion of the captured representation 50, that corresponds to the cutaneous condition 30.

In another example, the method 400 may include a fourth process 440 of rendering one or more portions of previously captured representations of the cutaneous condition within the enclosed area on the at least one display.

In another example, the method 400 may include a fifth process 450 of rendering one or more visual representations of previously determined sets of characteristics corresponding to the cutaneous condition within the enclosed area on the at least one display.

For example, in allowing the user to access previously determined sets of characteristics corresponding to the one or more cutaneous conditions, the LMA may render, on the at least one display, portions of previously captured representations corresponding to a cutaneous condition of the one or more cutaneous conditions. The LMA may render the portions of previously captured representations such that the portions of previously captured representations corresponding to temporally distinct mappings of the cutaneous condition may be rendered simultaneously.

In another example, the first process 410, the second process 420, the third process 430, the fourth process 440, and the fifth process 450 may occur simultaneously, for e.g., during the same evaluation of the cutaneous surface.

FIG. 11 depicts interactive prompts generated by the LMA according to some examples. Referring to FIG. 11, the LMA may also generate prompts on a present or later mapping(s) of the cutaneous surface, for the user to update any values and/or quantities including any notes or observations corresponding to the one or more cutaneous conditions of the cutaneous surface to encourage a complete ongoing record of the cutaneous conditions being monitored. In an illustrative example, the LMA 500 may cause the processor 10 to generate one or more prompts PR corresponding to the determined characteristics of the cutaneous condition 30 on the cutaneous surface 20.

In another example, the LMA may cause the at least one processor to render, on the at least one display, the one or more prompts generated by the LMA. In an illustrative example, the LMA 500 may cause the display 12 via the processor 10 to render the one or more prompts PR. A user of the LMA 500 may input, on the display 12, any values and/or quantities, including any notes or observations in response to the one or more prompts PR. The values and/or quantities inputted by the user in response to the one or more prompts PR may be stored in a central repository, for e.g., stored in cloud database 16, such that the inputted values and/or quantities may be accessed at a later time and/or location.

In another example, the prompts generated by the LMA on the display may be interactive, such that a user is able to manually interact with the prompts via the display, for e.g., via a capacitive-touch based mechanism of the display. In an illustrative example, the user may interact the display 12 to interact with the one or more prompts PR rendered on the display 12.

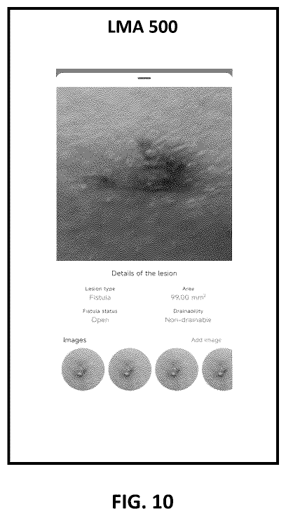

In another example, the LMA may allow a user to interact with the display, such that the user is able to access past assessments, including previously captured representations and previously determined characteristics, of the one or more cutaneous conditions of the cutaneous surface. In an illustrative example and with reference to FIG. 10, the LMA 500 may allow the user to interact with the display 12 to view information corresponding to previously conducted assessments, including previously captured representations and previously determined characteristics of the cutaneous condition 30. In further examples, the LMA 500 may allow the user to interact with the display 12 to edit information corresponding to previously conducted assessments, including previously captured representations and previously determined characteristics of the cutaneous condition 30.

In another example, the LMA may allow a user to interact with the display, such that the user may input information corresponding to the subject whose cutaneous surface is being mapped. For example, the LMA may allow a user to input information, including the birth date, medical history, and any other applicable information, that corresponds to the subject. The information inputted by the user of the LMA may be associated with a subject profile, such that any information associated with any and/or all mappings of the subject, including any determined characteristics of the one or more cutaneous conditions of the cutaneous surface of the subject, may be accessed at a later time and/or location.

In another example, The LMA, through the at least one processor, may apportion the total surface area of the cutaneous surface into one or more segments of the cutaneous surface (for e.g., the head, the right arm, the right leg, etc.). In further examples, a user of the LMA, for e.g., a clinician, may interact with the at least one display to select a segment of the one or more segments of the cutaneous surface. for mapping of the one or more cutaneous conditions thereon.

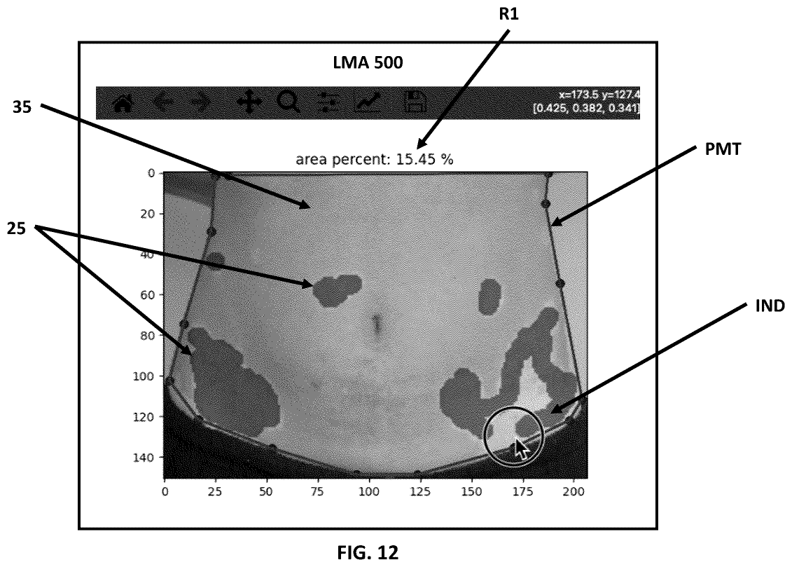

In some examples, and with respect to FIG. 12, the LMA may determine, via the at least one processor, a ratio based on the determined characteristics of the one or more cutaneous conditions and/or the determined characteristics of the one or more measurement markers.

In another example, the ratio determined by the LMA may be between a first characteristic corresponding to a first portion of the cutaneous surface with respect to a second characteristic corresponding to a second portion of the cutaneous surface. In an illustrative example, the LMA 500 may determine a ratio R1 between a first characteristic 80 corresponding to a first portion 90 of the cutaneous surface 25 with respect to a second characteristic 85 corresponding to a second portion 95 of the cutaneous surface 25.

In another example, the first characteristic may comprise a qualitative and/or quantitative value that corresponds to the first portion of the cutaneous condition. In further examples, the second characteristic may comprise a qualitative and/or quantitative value that corresponds to the second portion of the cutaneous condition.

In another example, the first portion and/or the second portion may comprise the entirety of the cutaneous surface being evaluated (for e.g., the entirety of the cutaneous surface of the patient being evaluated). In further examples, the first portion and/or the second portion may comprise portion(s) of the cutaneous surface shown in the captured representation.

In another example, the first portion and/or the second portion may comprise portion(s) of the cutaneous surface having the one or more cutaneous conditions. For example, the LMA may determine a ratio between the total surface area of the portion(s) of the cutaneous surface having the one or more cutaneous conditions with respect to the total surface area of the portion(s) of cutaneous surface shown in the captured representation.

In another example, the first portion and/or the second portion may be defined by the LMA through Artificial Intelligence/Machine Learning (“AI/ML”) algorithm(s) stored in the at least one memory and executed by the at least one processor. For example, the LMA may form, via the AI/ML algorithm(s), a first perimeter on the captured representation to define the first portion and a second perimeter on the captured representation to define the second portion.

In another example, the first portion and/or the second portion may be defined by the user of the LMA. In some examples, the user of the LMA may interact with the at least one display to place a perimeter on the captured image to respectively define the first portion and/or the second portion. In an illustrative example, the user of the LMA 500 may place a perimeter PMT around the cutaneous surface 25 to define the first portion 90.

In other examples, the user of the LMA may interact with the at least one display to manually define the first portion and/or the second portion of the cutaneous surface. In an illustrative example, the user of the LMA 500 may use a pointer, for e.g., a finger, on the display 12 to place an indicator IND corresponding to the respective surface areas of the cutaneous conditions 35 to define the second portion 95.

In another example, the first portion, the second portion, and/or a combination of both may be defined by the LMA and/or a user of the LMA. In an illustrative example, the user of the LMA 500 may place a perimeter PMT around the cutaneous surface 25 to define the first portion 90 and place an indicator IND corresponding to the respective surface areas of the cutaneous conditions 35 to define the second portion 95.

In further examples, the user may modify the first portion and/or the second portion previously defined by the LMA, for e.g., by interacting with the at least one display.

In an illustrative example, a user of the LMA 500 may define the first portion 90 to comprise the cutaneous surface 25 and the second portion 95 to comprise the cutaneous conditions 35, as depicted in FIG. 12. The user may further define the first characteristic 80 to correspond to the total surface area of the first portion 90 and the second characteristic 85 to correspond to the total surface area of the second portion 95. The LMA 500 may then contemporaneously determine, via the processor 10, the ratio R1 between the first characteristic 80 of the first portion 90 with respect to the second characteristic 85 of the second portion 95.

In this way, a user of the LMA, for e.g., a clinician, is able to determine the ratio between varying characteristics and/or portions of a patient's cutaneous surface, including a ratio between the total surface area of the patient's cutaneous surface affected by cutaneous conditions with respect to the total surface area of the patient's body unaffected by the cutaneous conditions (or the total surface area of the entirety of the patient's body including the affected portions), to aid in evaluating progression of the cutaneous conditions. Thus, the LMA allows the clinician to maintain accuracy and consistency in subsequent evaluations of the cutaneous conditions, even if preceding evaluations are conducted by other clinician(s).

It will be understood that the example embodiments described hereinabove are merely exemplary and that a person skilled in the art may make many variations and modifications without departing from the spirit and scope of the present invention. All such variations and modifications are intended to be included within the scope of the invention as defined in the accompanying claims.

What is claimed is:

1. A system for mapping one or more cutaneous conditions of a cutaneous surface, the system comprising:
at least one processor,
at least one display, the at least one display configured to receive user input, and operatively connected to the at least one processor;
at least one camera, the at least one camera operatively connected to the at least one processor;
a memory, the memory operatively connected to the at least one processor and storing instructions that, when executed, cause the at least one processor to perform a method, the method comprising:
receiving a captured representation of a cutaneous surface having one or more cutaneous conditions and one or more measurement markers;
defining one or more boundaries around the one or more cutaneous conditions on the captured representation to form one or more enclosed areas;
determining, based on a comparison between the one or more measurement markers and the one or more enclosed areas, characteristics of the one or more cutaneous conditions enclosed within the one or more enclosed areas; and
rendering, on the at least one display, the captured representation and the determined characteristics of the one or more cutaneous conditions enclosed within the one or more enclosed areas.

2. A system for mapping one or more cutaneous conditions of a cutaneous surface, the system comprising:
at least one processor,
at least one display, the at least one display configured to receive user input, and operatively connected to the at least one processor;
at least one remote sensor, the at least one remote sensor operatively connected to the at least one processor; and
a memory, the memory operatively connected to the at least one processor and storing instructions that, when executed, cause the at least one processor to perform a method, the method comprising:
receiving a captured representation of a cutaneous surface having one or more cutaneous conditions and one or more measurement markers;
defining one or more boundaries around the one or more cutaneous conditions on the captured representation to form one or more enclosed areas;
determining, based on a comparison between the one or more measurement markers and the one or more enclosed areas, characteristics of the one or more cutaneous conditions enclosed within the one or more enclosed areas; and
rendering, on the at least one display, the captured representation and the determined characteristics of the one or more cutaneous conditions enclosed within the one or more enclosed areas.

3. A method for mapping one or more cutaneous conditions of a cutaneous surface, the method comprising:
receiving, by at least one processor, a captured representation of a cutaneous surface having one or more cutaneous conditions and one or more measurement markers;
defining, by the at least one processor, one or more boundaries around the one or more cutaneous conditions on the captured representation to form one or more enclosed areas;
determining, by the at least one processor and based on a comparison between the one or more measurement markers and the one or more enclosed areas, characteristics of the one or more cutaneous conditions enclosed within the one or more enclosed areas; and
rendering, by the at least one processor on at least one display, the captured representation and the determined characteristics of the one or more cutaneous conditions enclosed within the one or more enclosed areas.
